(19) United States
(12) Patent Application Publication
(10) Pub. No.: US 2021/0111560 A1
(43) Pub. Date: Apr. 15, 2021

(54) Energy Management System and Energy Management Method

(71) Applicant: Hitachi, Ltd., Tokyo (JP)

(72) Inventor: Naoki YOSHIMOTO, Tokyo (JP)

(21) Appl. No.: 17/029,272

(22) Filed: Sep. 23, 2020

(30) Foreign Application Priority Data
Oct. 10, 2019 (JP) 2019-186930

(51) Int. Cl. H02J 3/14; G06Q 50/06; G06Q 10/06; G05B 19/042; H02J 13/00

(52) U.S. Cl. CPC H02J 3/14; G06Q 50/06; G06Q 10/06315; A61B 5/024; H02J 13/00006; H02J 13/00002; G05B 2219/2639; G05B 19/042

(57) ABSTRACT

Provided is an energy management system that appropriately grasps electric power demand prediction of the electric power in a business department, and implements electric power cooperation maintaining a business state. The energy management system includes a processor and a storage device. The storage device stores, in association with each other, past position information indicating a past position of a person in a target space and electric power demand information indicating an electric power demand in the target space when the past position information is acquired, and the processor searches for the past position information similar to new position information indicating a newly acquired position of the person in the target space, and outputs control information for controlling the electric power demand in the target space based on the electric power demand information stored in the storage device corresponding to the past position information obtained by the search.

### CLAIM OF PRIORITY

The present application claims priority from Japanese patent application JP 2019-186930 filed on Oct. 10, 2020, the content of which is hereby incorporated by reference into this application.

### BACKGROUND OF THE INVENTION

### 1. Field of the Invention

The present invention relates to an electric power supply and demand management technique.

### 2. Description of the Related Art

In recent years, the spread of renewable energy toward reduction of greenhouse gas is continuing. Among renewable energies, variable renewable energy (VRE), especially wind electric power and solar electric power, is pointed out to be necessary to adjust a supply and demand balance in a form that does not rely on fossil fuel-based electric power generation such as thermal electric power generation in the related art due to fluctuations in energy supply.

Among electric power demands, business departments such as office buildings, commercial facilities, and public facilities recently have an increasing trend of a ratio of electric power demand to the whole in recent years. Since electric power demand characteristics in the business department are that consumers are diverse, there are also various electric power consumption trends, and it is difficult to predict the demand.

Regarding grasp of the electric power demand in the business department, a method of schedule-registering a working time zone and event information of the relevant business department, and planning an electric power demand based on the registered schedule (JP-A-2017-227999 (PTL 1)), and a method of giving information on weather and temperature by a weather forecast (JP-A-2017-169418 (PTL 2)) in addition to the schedule-based electric power demand planning are studied in the related art.

Further, in order to adjust the supply and demand balance of the electric power, not only the electric power demand but also a supply prediction technique of the variable renewable energy (VRE) is studied (JP-A-2002-135977 (PTL 3)).

As described above, the demand prediction of the electric power in the business department in the related art generally corresponds to the scheduled-based electric power demand. It is difficult to deal with a duck curve near the evening, or to deal in detail with the adjustment of supply and demand for each tenant.

In the business apartment, there are various uses such as office buildings, complex buildings that combine commercial facilities and offices, commercial facilities such as large shopping malls, public facilities such as city halls and cultural halls, and thus the electric power demand for each use also has various characteristics. Considering the diversification of lifestyle and working environment, there is a limit to meeting the schedule-based electric power demand. At the same time, it is difficult to meet the electric power demand by simply managing the schedule in order to minimize the influence on the living and working environment of the consumer.

### SUMMARY OF THE INVENTION

In order to solve at least one of the problems described above, the invention relates to an energy management system including a processor and a storage device. The storage device stores, in association with each other, past position information indicating a past position of a person in a target space and electric power demand information indicating an electric power demand in the target space when the past position information is acquired, and the processor searches for the past position information similar to new position information indicating a newly acquired position of the person in the target space, and outputs control information for controlling the electric power demand in the target space based on the electric power demand information stored in the storage device corresponding to the past position information obtained by the search.

According to the invention, the electric power demand of a consumer and the electric power supply including variable renewable energy (VRE) can be coordinated with each other.

Problems, configurations, and effects other than those described above will be clarified by the following description of embodiments.

### BRIEF DESCRIPTION OF THE DRAWINGS

FIG. 1 is a block diagram showing a configuration of an indoor energy supply and demand management apparatus constituting an energy management system according to an embodiment of the invention.

FIG. 2 is an explanatory diagram of an overall configuration of the energy management system and a hardware configuration of the indoor energy supply and demand management apparatus according to the embodiment of the invention.

FIG. 3 is an explanatory diagram showing an example of a layout of a space managed by the indoor energy supply and demand management apparatus according to the embodiment of the invention.

FIG. 4 is an explanatory diagram showing an example of a movement of a person in the space managed by the indoor energy supply and demand management apparatus according to the embodiment of the invention.

FIG. 5 is an explanatory diagram of a processing data storage unit of the indoor energy supply and demand management apparatus according to the embodiment of the invention.

FIG. 6 is an explanatory diagram showing an overall processing flow of the energy management system according to the embodiment of the invention.

FIG. 7 is an explanatory diagram showing a processing flow of electric power demand verification in a position space performed by the indoor energy supply and demand management apparatus according to the embodiment of the invention.

FIG. 8 is an explanatory diagram showing a processing flow of similarity verification with accumulation data of position and movement information of the person and an electric power demand situation performed by the indoor energy supply and demand management apparatus according to the embodiment of the invention.

### DESCRIPTION OF THE PREFERRED EMBODIMENTS

An embodiment of the invention will be described below. The following embodiment is an example, and the invention is not limited to the embodiment.

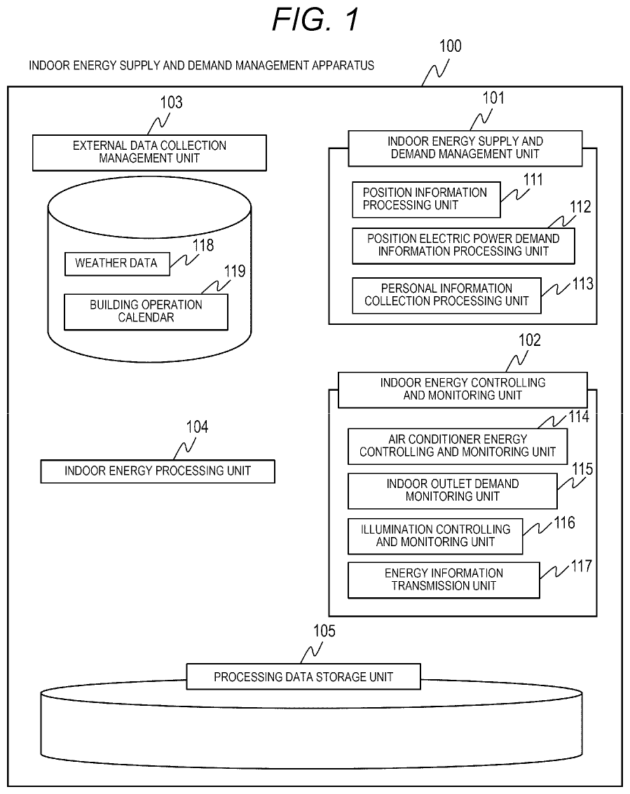

FIG. 1 is a block diagram showing a configuration of an indoor energy supply and demand management apparatus 100 constituting an energy management system according to an embodiment of the invention.

The indoor energy supply and demand management apparatus 100 includes an indoor energy supply and demand management unit 101, an indoor energy controlling and monitoring unit 102, an external data collection management unit 103, an indoor energy processing unit 104, and a processing data storage unit 105.

The indoor energy supply and demand management unit 101 manages electric power supply and demand in a space (for example, in an office room) to be managed by the indoor energy supply and demand management apparatus 100. The indoor energy supply and demand management unit 101 shown in FIG. 1 includes a position information processing unit 111 for acquiring position information of a person indoors, a position electric power demand information processing unit 112 for processing electric power demand information for each indoor position, and a personal information collection processing unit 113 for collecting personal information of the indoor person. Details of functions these units will be described later.

In the present embodiment, a space in one floor of the office is mainly shown as an example of the space to be managed by the indoor energy supply and demand management apparatus 100. The space to be managed is not limited thereto, and may be, for example, a commercial facility including one or more stores, or an individual residence. In addition, an optional type of space may be the space to be managed by the indoor energy supply and demand management apparatus 100 according to the present embodiment.

The indoor energy controlling and monitoring unit 102 controls indoor energy, for example, controls electric power supply or controls and monitors devices that consumes electric power. The indoor energy controlling and monitoring unit 102 shown in FIG. 1 includes an air conditioner energy controlling and monitoring unit 114 for controlling and monitoring an air conditioner configured to adjust an indoor temperature and the like, an indoor outlet demand monitoring unit 115 for monitoring a demand for an outlet (that is, an outlet or a socket) for supplying the electric power to indoor electric devices, an illumination controlling and monitoring unit 116 for controlling and monitoring an illumination device configured to illuminate the room, and an energy information transmission unit 117 for outputting information related to energy. Details of functions of these units will be described later.

The external data collection management unit 103 collects external data and stores the collected external data into the indoor energy supply and demand management apparatus 100. The collected external data includes, for example, weather data 118 and a building operation calendar 119. The weather data 118 may include, for example, data such as weather, outside air temperature, and humidity in a region including the space to be managed. The building operation calendar 119 is a schedule of events in the space to be managed. For example, the building operation calendar 119 may include information such as a business day of the office, business hours, and a day when a special event is held, when the space to be managed is a commercial facility such as a store; may include information such as the business day and special sale date of the store; and when the space to be managed is an individual residence, may include information such as a scheduled home date of the resident and a schedule action of the resident while at home.

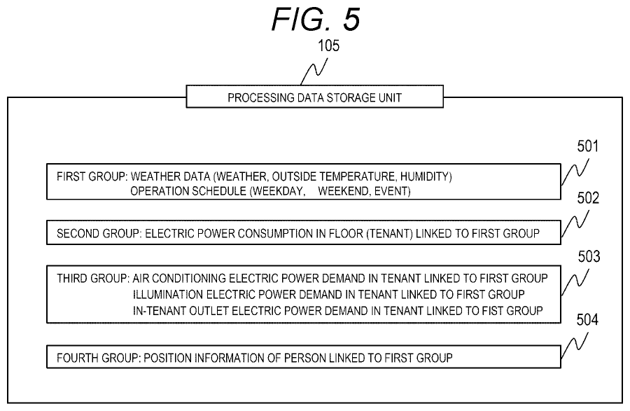

The processing data storage unit 105 stores data used for processing of the indoor energy supply and demand management apparatus 100, data obtained as a result of the processing, and the like. Details thereof will be described later (see FIG. 5).

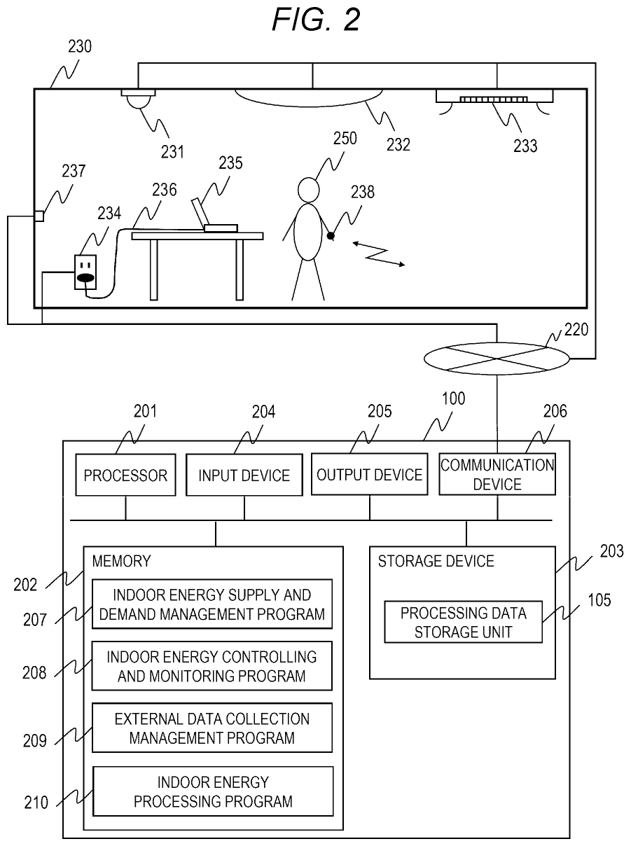

FIG. 2 is an explanatory diagram of an overall configuration of the energy management system (region energy management system) and a hardware configuration of the indoor energy supply and demand management apparatus 100 according to the embodiment of the invention.

An energy management system 200 according to the present embodiment includes the indoor energy supply and demand management apparatus 100, various devices installed in a space 230 to be managed, and a network 220 connecting the above devices in a communicable manner.

The space 230 to be managed is, for example, an office space, and various kinds of furniture and the like are installed therein. For example, a position sensor 231, an illumination device 232, an air conditioner 233, an outlet 234, a personal computer (PC) 235, an environment sensor 237, and a personal information sensor 238 are installed in the space 230 to be managed. Although the above furniture is shown one by one in FIG. 2, a plurality of the furniture are generally installed in the space 230 in practice.

The position sensor 231 may be of any type as long as it can detect the position of a person 250 in the space 230. An example of the position sensor 231 is a human sensor for security using infrared rays. In this case, it is possible to detect the number of persons 250 within a predetermined distance from the position sensor 231. It is desirable to install a sufficient number of position sensors 231 to be capable of detecting all persons 250 in the space 230.

Here, the person 250 is a person who performs an action of consuming electric power in the space 230 (that is, a consumer). For example, if the space 230 is an office, the person 250 may be an office worker in the office. If the space 230 is a store, the person 250 may be a customer or an employee of the store. If the space 230 is a residence, the person 250 may be a resident of the residence.

Each position sensor 231 can transmit detection result data to the indoor energy supply and demand management apparatus 100 via the network 220. The indoor energy supply and demand management apparatus 100 can estimate the number of persons 250 in a certain area of the space 230 based on an installation position of each position sensor 231 and the number of persons detected by each position sensor 231.

The illumination device 232 is an electric device that illuminates the space 230, such as a fluorescent lamp or a light emitting diode (LED) lamp. A plurality of illumination devices 232 are installed in the space 230. Each illumination device 232 can control illumination intensity based on control information from the indoor energy supply and demand management apparatus 100. Each illumination device 232 may have a function of controlling the illumination intensity based on the control information obtained by communicating with the indoor energy supply and demand management apparatus 100. Alternatively, the illumination intensity may be controlled by controlling electric power supply to each illumination device 232 based on the control information from the indoor energy supply and demand management apparatus 100. The illumination devices 232 may be independently controlled, and may be controlled for each area within the space 230.

The air conditioner 233 is an electric device that adjusts the temperature of the air in the space 230. A plurality of air conditioners 233 are installed in the space 230, and temperature adjustment intensity can be controlled based on the control information from the indoor energy supply and demand management apparatus 100. The control may be performed by the function of the air conditioner 233, or may be performed by controlling the electric power supply to the air conditioner 233, as in the case of the illumination device 232. The air conditioners 233 may be independently controlled, and may be controlled for each area within the space 230.

The PC 235 is, for example, an information processing apparatus used for the work of the person 250, and is treated as an electric device that consumes the electric power similar to the illumination device 232 and the like in the present embodiment. The electric power is supplied from the outlet 234 to the PC 235 via a cable 236. The outlet 234 is connected to the indoor energy supply and demand management apparatus 100 via the network 220, and can control (for example, supply or cut off) the electric power supply based on the control information from the indoor energy supply and demand management apparatus 100. If the PC 235 has a built-in battery, the PC 235 can be continuously used for awhile even when the electric power supply from the outlet 234 is cut off.

The environment sensor 237 may include at least one of a temperature sensor, a humidity sensor, and an illuminance sensor, for example. A measurement result of the sensor is transmitted to the indoor energy supply and demand management apparatus 100 via the network 220. In general, since the air conditioner 233 includes a temperature sensor or the like, the indoor energy supply and demand management apparatus 100 may obtain information such as the temperature therefrom, or may use the environment sensor 237 installed at a position close to the person 250 in order to obtain more accurate information.

The personal information sensor 238 acquires personal information of the person 250 and transmits the result to the indoor energy supply and demand management apparatus 100 via the network 220 by wireless or wired communication. Here, the personal information of the person 250 may include biological information of the person 250 (for example, a pulse), or an activity amount (for example, acceleration of the body of the person 250 or an action of the person 250 estimated in accordance with the acceleration), or a result of detecting a meeting or conversation with another person.

The indoor energy supply and demand management apparatus 100 according to the present embodiment includes a processor 201, a memory 202, a storage device 203, an input device 204, an output device 205, and a communication device 206.

The processor 201 controls the indoor energy supply and demand management apparatus 100 in accordance with a program stored in the memory 202.

The memory 202 is, for example, a semiconductor memory, and stores a program to be executed by the processor 201, data to be referred to by the processor 201, and data acquired as a result of processing executed by the processor 201. At least a part of program and data stored in the storage device 203 may be copied to the memory 202 as necessary, or the acquired data may be copied from the memory 202 to the storage device 203 as necessary.

In the example of FIG. 2, an indoor energy supply and demand management program 207, an indoor energy controlling and monitoring program 208, an external data collection management program 209, and an indoor energy processing program 210 are stored in the memory 202. These are programs for implementing processing of the indoor energy supply and demand management unit 101, the indoor energy controlling and monitoring unit 102, the external data collection management unit 103, and the indoor energy processing unit 104 shown in FIG. 1. In the present embodiment, the processing executed by each unit is actually executed by the processor 201 in accordance with instructions described in the programs.

The storage device 203 includes a nonvolatile storage medium such as a flash memory or a hard disk. The processing data storage unit 105 shown in FIG. 1 corresponds to a storage region of the storage device 203.

The input device 204 receives an input from a user of the indoor energy supply and demand management apparatus 100. Specifically, the input device 204 may include, for example, a keyboard, buttons, or a pointing device. For example, at least a part of the information stored in the storage device 203 may be input via the input device 204.

The output device 205 outputs information to the user of the indoor energy supply and demand management apparatus 100. Specifically, the output device 205 may include an image display device, for example.

The communication device 206 is a device for enabling exchange of data between the indoor energy supply and demand management apparatus 100 and an external device (for example, an electric device installed in the space 230 to be managed) via a wired or wireless network, and may include, for example, a local area network (LAN) adapter. For example, at least a part of the information stored in the storage device 203 may be input via the communication device 206, or at least a part of the information generated as a result of the processing according to the program may be output via the communication device 206.

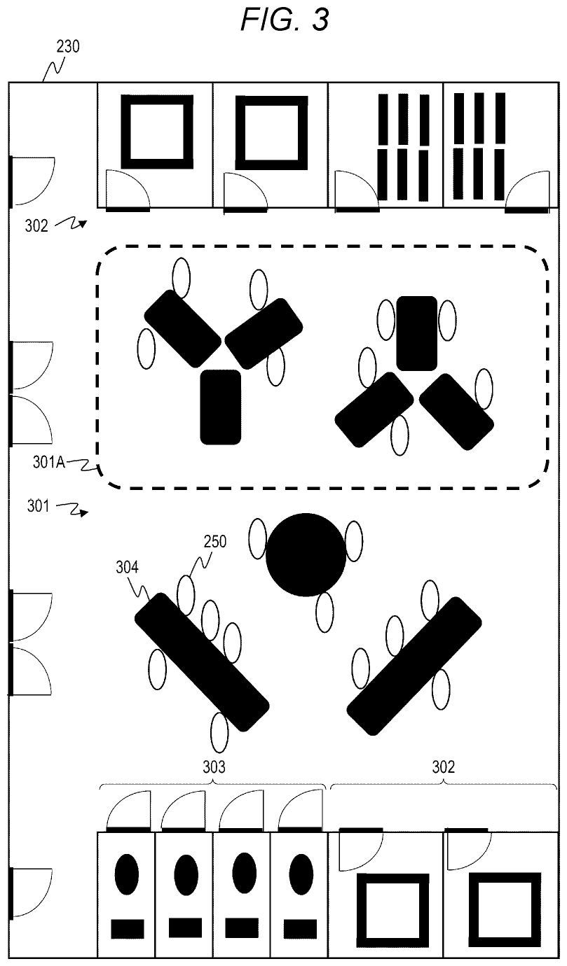

FIG. 3 is an explanatory diagram showing an example of a layout of the space 230 managed by the indoor energy supply and demand management apparatus 100 according to the embodiment of the invention.

FIG. 3 is a plan view of a floor of a so-called free address office as an example of the space 230.

A multi-purpose area 301 in which a plurality of tables 304, chairs (not shown), and the like are arranged is provided in the center of the space 230 shown in FIG. 3. The area can be used for multiple purposes, such as meetings and individual desk work. The multi-purpose area may be further divided into a plurality of areas. These areas may or may not be separated by a partition or the like (not shown).

A plurality of conference rooms 302 are provided at one end of the space 230 (upper portion in FIG. 3). These are areas used for, for example, a conference, and are separated from other areas by partitions, doors, and the like. A plurality of conference rooms 302 and a plurality of private rooms 303 are provided at the other end of the space 230 (lower portion in FIG. 3). The private room is an area used by each person 250 to perform work using, for example, the PC 235 and is separated from other areas by partitions, doors, and the like.

A plurality of illumination devices 232, a plurality of air conditioners 233, and a plurality of outlets 234 (all not shown in FIG. 3) are installed in the space 230 in order to implement the electric power supply for the illumination and air conditioning and to the electric devices being used in each area. Further, a plurality of position sensors 231 and environment sensors 237 (both not shown in FIG. 3) are installed in the space 230. Each electric device and each sensor and each area may not be associated with each other in a one-to-one correspondence. For example, one air conditioner 233 may be responsible for the air conditioning of a plurality of private rooms, or a plurality of illumination devices may be responsible for illumination of one conference room, or a plurality of position sensors 231 may be responsible for one multi-purpose area 301. The same applies to the outlet 234. The indoor energy supply and demand management apparatus 100 stores information indicating a correspondence between each electric device, each sensor, and each outlet, and the corresponding responsible area thereof.

Each of a plurality of white ellipses shown in FIG. 3 indicates one person 250. In the example of FIG. 3, a plurality of persons 250 are in the multi-purpose area 301. Although omitted in FIG. 3, each person 250 may hold the personal information sensor 238.

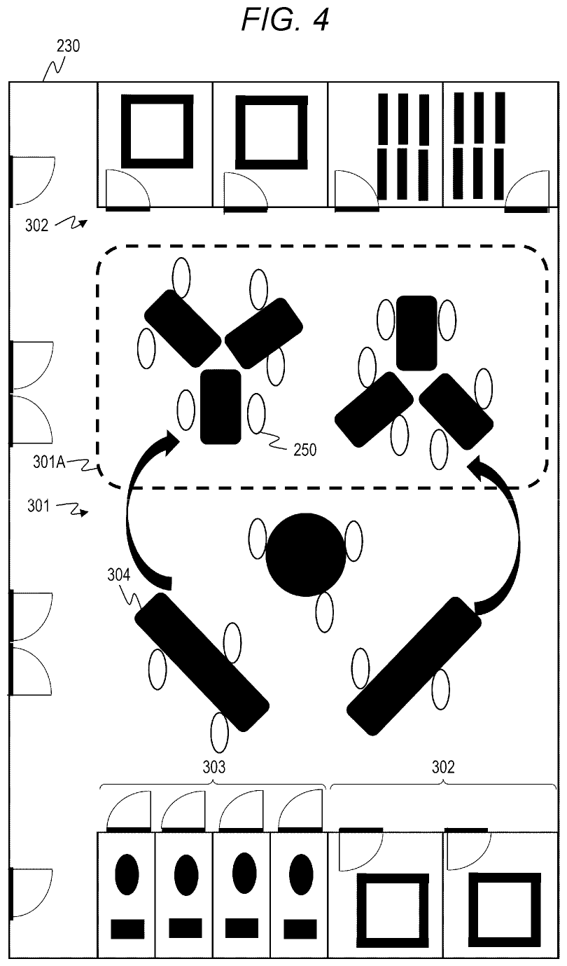

FIG. 4 is an explanatory diagram showing an example of a movement of the person 250 in the space 230 managed by the indoor energy supply and demand management apparatus 100 according to the embodiment of the invention.

FIG. 4 shows an example in which the movement of the person 250 occurs in the space shown in FIG. 3. FIG. 4 shows a situation of the space 230 at a time later than the time when the position of the person 250 shown in FIG. 3 is observed. In this example, three persons in an area other than an area 301A surrounded by a broken line in the multi-purpose area 301 move into the area 301A, and one person leaves the space 230. As a result, as compared with a time point of FIG. 3, at the time of FIG. 4, the number of persons 250 in the area 301A of the multi-purpose area 301 increases, and the number of persons 250 in other areas decreases.

The indoor energy supply and demand management apparatus 100 can estimate the number of persons 250 in each area at each time based on data obtained from each position sensor 231 at each time. Further, when the number of persons 250 is changed by comparing these pieces of data, it is possible to estimate which person 250 in which area moves to which area.

FIG. 5 is an explanatory diagram of the processing data storage unit 105 of the indoor energy supply and demand management apparatus 100 according to the embodiment of the invention.

The processing data storage unit 105 stores a first group of data 501, a second group of data 502, a third group of data 503, and a fourth group of data 504.

The first group of data 501 includes weather data and an operation schedule collected by the external data collection management unit 103. The second group of data 502 includes electric power consumption in the space 230. The electric power consumption is associated with the weather data and the operation schedule at the time of acquisition.

The third group of data 503 includes electric power demands of the illumination device 232, the air conditioner 233, and the electric device connected to the outlet 234 in the space 230. The electric power demands are associated with the weather data and the operation schedule at the time of acquisition.

The fourth group of data 504 includes position information of the person 250 in the space 230. The position information may be, for example, information such as the number of persons 250 in a certain area at a certain time. The position information is associated with the weather data and the operation schedule at the time of acquisition.

In addition, the processing data storage unit 105 may include position information of furniture (for example, desks, tables, chairs, whiteboards, and document cabinets) in the space 230, or may include information for estimating an action of the person 250 based on a relationship between the position of the person 250 and the position of the furniture.

### (System Processing Flow)

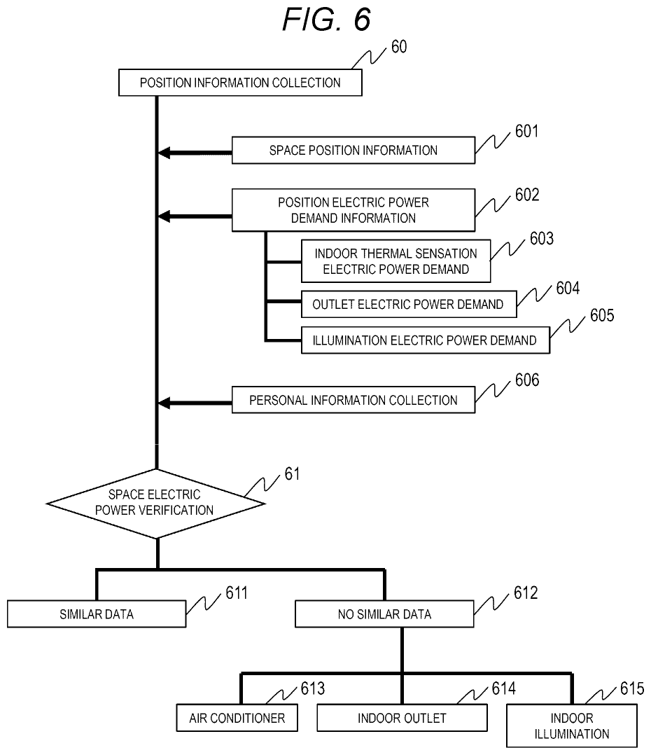

FIG. 6 is an explanatory diagram showing an overall processing flow of the energy management system 200 according to the embodiment of the invention.

The processing of the energy management system 200 of the present embodiment includes a position information collection step 60 and a space verification step 61, and constitutes a basic configuration of the present embodiment.

### (Position Information Collection Step 60)

In the position information collection step 60, the indoor energy supply and demand management apparatus 100 first collects preset information related to interior of an office floor. Accordingly, for example, position information of furniture such as desks, tables, chairs, and partitions, and position information of areas such as a conference room area and a private room when these areas are set, are collected. This step may be performed at the time of changing the layout or the like of the interior, and does not need to be performed all the time.

According to the present embodiment, the electric power demand of the consumer is grasped and predicted based on the position information of the consumer. Therefore, it is desirable that the interior is arranged to reflect the living and working environment of the consumer. In recent years, the office interior environment is established as a free address office where the resident selects from various interiors and environments to work, not a layout where the furniture is in a fixed position and a seating place is fixed as in the related art. By providing an environment for workers to select, such as the free address office, there is a correlation between a trend of the energy demand and living position and working position of the worker, so that the correlation can be used for the electric power demand. Accordingly, it is desirable, but not limited, to apply the interior environment that allows diversity, such as the free address office, as the office interior environment.

In the position information collection step 60, the indoor energy supply and demand management apparatus 100 then collects space position information 601 of the person 250 who is the consumer. The space position information 601 of the person 250 is important information corresponding to the energy demand in the office interior environment based on the living environment or the working environment of the person 250. In order to grasp the energy demand situation of the person 250, it is necessary to grasp a temporal change of the space position of the person 250 in the office interior environment. Therefore, the indoor energy supply and demand management apparatus 100 grasps the position information of the person 250 at each time in order to grasp the situation of the person 250 such as work performed while the person 250 stays in the office, the movement of the person 250 in the office, and work of the person 250 using the space in the office. A grasp period for each time of the position information of the person 250 is desirably about one minute. When the period is shorter than one minute, data amount becomes enormous and a system load increases, and on the contrary, when the period is three minutes or longer, it is difficult to determine the stay and movement of the person 25 in the space, which is not preferable.

A method of acquiring the space position information 601 of the person 250 is not particularly limited. In order to acquire the space position information 601, a security facility such as a security purpose camera or a heat ray sensor is used in combination, thereby reducing an equipment introduction cost.

As a first requirement of the space position information 601 of the person 250, two-dimensional position information of the person 250 indoors is exemplified. According to the present embodiment, since there is a correlation between the energy demand and the indoor living condition, it is possible to grasp the energy demand by the two-dimensional position information of the consumer.

As a second requirement of the space position information 601 of the person 250, two-dimensional movement information of the person 250 indoors is exemplified. According to the present embodiment, there is a correlation between the energy demand and the movement state and movement form of the person 250 in the indoor space. By grasping the movement form of the resident indoors, it is possible to grasp the energy demand required by the person 250. The movement state and the movement form of the person 250 indoors can be grasped by obtaining a temporal change of the two-dimensional position information.

As a second element of the position information collection step, the indoor energy supply and demand management apparatus 100 collects position electric power demand information 602 of the person 250. The electric power demand information of the person 250 can be roughly divided into (1) indoor thermal sensation control energy demand 603 such as air conditioning, airflow and ventilation, (2) outlet electric power demand 604 required for indoor business execution, and (3) indoor illumination electric power demand 605. Information of the three elements may be collected.

As the position electric power demand information 602 of the person 250, information about all elements of the thermal sensation control energy demand 603, the outlet electric power demand 604, and the illumination electric power demand 605 are preferably collected. However, when the installation of sensors and measurement instruments for collecting information such as existing buildings is insufficient, it is possible to fulfill the function by using a part of information of one or more of the three elements.

Among the position electric power demand information 602 of the person 250, information of the indoor thermal sensation control energy demand 603 such as air conditioning, airflow and ventilation can be collected by various existing methods such as a method of referring to a control state of the air conditioner 233. For example, it is possible to grasp individual output of a blowout port according to an operation situation of a package air conditioner (PAC). According to the present embodiment, in order to grasp the electric power demand based on the position information of the person 250, it is desirable to collect as much electric power demand information as possible related to the position of the person 250. Therefore, it is more preferable, but not essential, not only to acquire the control situation of the air conditioner 233, but to use the environment sensor 237 or the like to separately acquire the indoor temperature, humidity, and, in some cases, a mean radiant temperature (MRT).

Among the position electric power demand information 602 of the person 250, the outlet electric power demand 604 required for the indoor business execution or the like may be acquired by various existing methods, and the acquisition method is not particularly limited. In general, an indoor outlet power supply is often limited to acquiring data for each room. Therefore, it is more preferable, but not essential, to obtain an outlet electric power demand that can be more correlated with individual business forms, such as electric power consumption per OA tap.

Among the position electric power demand information 602 of the person 250, the indoor illumination electric power demand 605 may be acquired by various existing methods, and the acquisition method is not particularly limited. However, in general, the electric power consumption of the illumination device 232 is rarely acquired individually, and is often treated collectively as indoor electric power consumption. In such a case, it is found from various past studies that an illuminance meter or the like is installed indoors to indirectly estimate the electric power consumption, and it is more preferable to indirectly associate the position information with the illumination electric power demand.

As a third element of the position information collection step, biological information 606 of the person 250 is exemplified. According to the invention, presence of the space position information and the position electric power demand information of the person 250 is sufficient as a necessary condition for the position information, and thus the biological information of the person 250 is not necessarily required. Although it is clarified by the past studies that the biological information of the person 250 is closely related to the indoor thermal sensation, the illumination environment, and the working situation, it is not essential to the present embodiment.

A collection period of the position information collection 60 is preferably about one minute. Although there is no problem in collecting information in a short period of one minute or shorter, data cleansing and decimation during data accumulation are required since the data amount increases. On the other hand, when the collection period is one minute or longer, the correlation between the movement state of the resident and the position electric power demand information is unclear, which is not preferable.

### (Electric Power Demand Verification 61 in Position Space)

Following the above position information collection, the indoor energy supply and demand management apparatus 100 performs the electric power demand verification 61 in the position space. The electric power demand verification 61 in the position space is connected with the electric power demand control in the position space through seven steps, including

(1) grasp of position information of the resident in the space,

(2) grasp of the movement situation of the resident in the space,

(3) correlation between a predetermined interior space information and position information of the resident,

(4) correlation between the predetermined interior space information and movement information of the resident,

(5) correlation between the predetermined interior space information, the position and movement information of the resident, and an electric power demand situation,

(6) similarity verification with accumulation data of the predetermined interior space information, the position and movement information of the resident, and the electric power demand situation, and

(7) a data accumulation step for the predetermined interior space information, the position and movement information of the resident, and the electric power demand situation.

The electric power demand verification 61 in the position space will be described below.

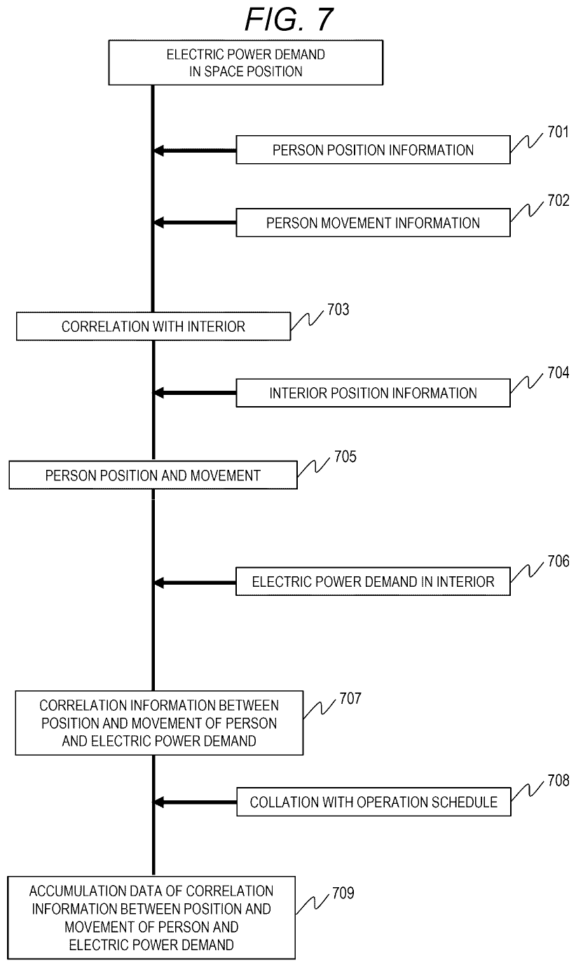

FIG. 7 is an explanatory diagram showing a processing flow of the electric power demand verification 61 in the position space performed by the indoor energy supply and demand management apparatus 100 according to the embodiment of the invention.

The electric power demand verification in the position space shown in FIG. 7 relates to a case where the space 230 is a floor of an office building, and is merely an example of the invention. When the invention is applied to a space other than the office building (for example, a store or a residence), there is no limitation to individual contents shown in FIG. 7.

As a first stage of the electric power demand verification 61 in the position space, the indoor energy supply and demand management apparatus 100 performs the (1) grasp of position information 701 of the person 250 in the space. According to the present embodiment, it is preferable that the predetermined interior space is a space that is characterized by the stay and movement of the person 250, including a free address office. In other words, it is desirable that there is a correlation between the area in the space 230 and the action of the person 250 staying in the area. For example, such a correlation is generated by preparing an area in the space 230 that has a certain purpose of use, such as a private room for PC work and a conference room for meetings. The indoor energy supply and demand management apparatus 100 grasps the position information of the person 250 in the space. The person 250 stays in a space according to his/her purpose and works therein. By grasping the correlation between the purpose and the energy demand, it is possible to grasp and predict the electric power demand of the consumer.

Subsequently, as a second stage, the indoor energy supply and demand management apparatus 100 performs the (2) grasp of a movement situation 702 of the person 250 in the space. According to the invention, there is a correlation between the energy demand and the movement state and the movement form of the person 250 in the indoor space. By grasping the movement form of the person 250 indoors, it is possible to grasp the energy demand required by the consumer. The movement state and the movement form of the person 250 indoors can be grasped by the temporal change of the two-dimensional position information. A grasp period for each time of the position information of the person 250 is desirably about one minute. When the period is shorter than one minute, the data amount becomes enormous, and a system load increases, and on the contrary, when the period is three minutes or longer, it is difficult to determine the stay and movement in the space, which is not preferable.

As a third stage, the indoor energy supply and demand management apparatus 100 performs (1) the grasp of the position information of the person 250 in the space, and (2) the grasp of the movement situation of the person 250 in the space, and, based on the result, (3) the grasp of the correlation between predetermined interior space information 704 and the position information of the person 250, and (4) the grasp of the correlation between the predetermined interior space information 704 and the movement information of the person 250 (step 703).

(3) The grasp of the correlation between the predetermined interior space information and the position information of the person 250 can be implemented by expressing (1) the grasp of the position information of the person 250 in the space in the predetermined interior space information two-dimensionally by any method. Further, (4) the grasp of the correlation between the predetermined interior space information and the movement information of the person 250 can be implemented by displaying (2) the grasp of the movement situation of the person 250 in the space in the predetermined interior space information by any method.

(3) The grasp of the correlation between the predetermined interior space information and the position information of the person 250 is performed as follows, for example. That is, for the purpose of executing business such as so-called free address office, it is desirable to provide a space where furniture, equipment, devices and illumination are provided to support the execution thereof, and the position information of the person 250 in such a space is highly likely to reflect the purpose of the action of the person 250. Therefore, by accumulating data of the correlation between the predetermined interior space information and the position information of the person 250, it is possible to grasp the outline of the action of the person 250 in this space.

(4) The grasp of the correlation between the predetermined interior space information and the movement information of the person 250 is performed as follows, for example. That is, for the purpose of executing business such as so-called free address office or wellness office considering the health of the resident, it is desirable to provide a space where furniture, equipment, devices and illumination are provided to support the execution thereof, and in the correlation with the movement information of the person 250 in such a space, it is highly likely that the sensitivity to the thermal sensation is correlated with the movement of the person 250 or the communication between the persons 250. Therefore, by accumulating the data of the correlation between the predetermined interior space information and the movement information of the person 250, it is possible to grasp the outline of the action of the person 250 in this space.

Then, (5) the grasp of the correlation between the predetermined interior space information, the position and movement information of the person 250, and an electric power demand situation 706 (step 707) will be described. It is highly possible that the thus obtained predetermined interior space information and position information and movement information (hereinafter, referred to as position and movement information) of the person 250 reflects the business state, which is the purpose of the person 250, and the movement of the person 250 and the sensitivity to the thermal sensation according to the communication between the persons 250. Therefore, it is highly possible that the grasp of the electric power demand at this time is to grasp the electric power demand that reflects the business state, which is the purpose of the person 250, and the sensitivity to the thermal sensation according to the movement of the person 250 and the communication between the persons 250. On the other hand, since the correlation between the predetermined interior space information, the position and movement information of the person 250, and the electric power demand situation is strongly influenced by the situation of the person 250, it is difficult to make a unique determination based on the predetermined interior space information. Therefore, it is necessary to grasp, by data accumulation, the correlation between the predetermined interior space information, the position and movement information of the person 250, and the electric power demand situation.

Next, (6) similarity verification with accumulation data of the predetermined interior space information, the position and movement information of the person 250, and the electric power demand situation, and (7) a data accumulation step 709 for the predetermined interior space information, the position and movement information of the person 250, and the electric power demand situation will be described. It is desirable that the accumulation data of the predetermined interior space information, the position and movement information of the person 250, and the electric power demand situation may be managed in categories such as a day of a week, time of day, and events such as schedule management of building equipment operation in the related art. Although there are no particular restrictions on the category of data management, the schedule management of the building equipment operation in the related art is a basic operation criterion of air conditioning equipment that manages the energy demand of more than 30% of the energy in an office building, so that it is effective to make use of correlation with the schedule management (step 708).

The indoor energy supply and demand management apparatus 100 verifies similarity between accumulation data managed in each category and the accumulation data of the current predetermined interior space information, the position and movement information of the person 250, and the electric power demand situation. If the similarity between data is recognized, in order to control the electric power supply based on the data, a difference between electric power demand data in the accumulation data and current electric power demand data is grasped. When an excess of the electric power demand is detected, the electric power supply is tried to be reduced.

Here, the similarity verification with the accumulation data of the position and movement information of the person 250 and the electric power demand situation will be described.

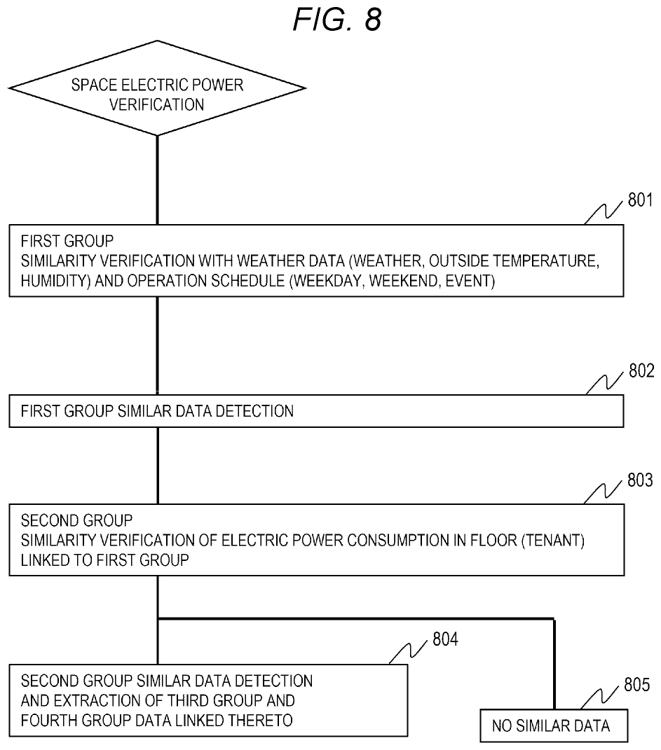

FIG. 8 is an explanatory diagram showing a processing flow of the similarity verification with the accumulation data of the position and movement information of the person 250 and the electric power demand situation performed by the indoor energy supply and demand management apparatus 100 according to the embodiment of the invention.

First, the indoor energy supply and demand management apparatus 100 verifies similarity between the current weather data and the operation schedule, and the first group of data 501 stored in the processing data storage unit 105 (step 801). Then, the indoor energy supply and demand management apparatus 100 detects the first group of data 501 similar to the current weather data and the operation schedule (step 802).

Next, the indoor energy supply and demand management apparatus 100 verifies the similarity between the electric power consumption in the space 230 corresponding to the first group of data 501 detected in step 802 among the second group of data 502 and the current electric power consumption in the space 230 (step 803). Then, when the indoor energy supply and demand management apparatus 100 detects electric power consumption similar to the current electric power consumption in the space 230 among the electric power consumption in the space 230 of the second group of data 502, the third group of data 503 and the fourth group of data 504 corresponding to the detected electric power consumption are extracted (step 804).

On the other hand, when the indoor energy supply and demand management apparatus 100 does not detect electric power consumption similar to the current electric power consumption in the space 230 among the electric power consumption in the space 230 of the second group of data 502, it is determined that there is no similar data (step 805).

Through the steps described above, the electric power demand verification 61 in the position space can be performed.

Subsequently, control of the electric power demand in the position space is performed. Although the following description takes the office building as an example, the invention is not limited to the office building, and can be used in various indoor living spaces.

According to the present embodiment, by performing the electric power demand verification 61 in the position space, it is possible to grasp the electric power demand of the person 250 at the space position. The electric power demand is roughly divided into three types: (1) air conditioner, (2) interior power supply, and (3) illumination. In the step of the electric power demand verification 61 in the position space, it is desirable that the accumulation data of the electric power demand situation is managed by being divided into categories such as the day of the week, time of day, and events such as the schedule management of the building equipment operation in the related art.

In the control of the electric power demand in the position space, actual control determination is performed as follows. First, in the electric power demand verification 61 in the position space, the indoor energy supply and demand management apparatus 100 verifies similarity between the accumulation data managed in each category and the accumulation data of the current predetermined interior space information, the position and movement information of the person 250, and the electric power demand situation. If there is a similar category case (step 611 in FIG. 6), the control is performed based on the accumulation data of that category.

In the present embodiment, when the similarity between the accumulation data managed by each category and the accumulation data of the current predetermined interior space information, the position and movement information of the person 250, and the electric power demand situation is recognized, it is also possible to grasp an electric power demand summary for a short time of about 30 to 60 minutes from the current electric power demand based on a trend of the accumulation data.

In the electric power demand verification 61 in the position space, the similarity between the accumulation data managed in each category and the accumulation data of the current predetermined interior space information, the position and movement information of the person 250, and the electric power demand situation is verified. If no similarity is found (step 612), electric power demand reduction is tried as follows. First, among (1) air conditioner, (2) interior power supply, and (3) illumination, the consumer reports in advance an order in which the electric power is to be adjusted, and the control for reducing the electric power demand is performed in the order (steps 613 to 615). In general, (2) the interior power supply is the easiest to adjust and it is easy to check with the consumer, so that the interior power supply is controlled first.

The control of the interior power supply is performed in two stages: first, information transmission of electric power reduction to the consumer (for example, the person 250), and subsequent electric power demand control including two types of electric power reduction, i.e., consumer participation type electric power reduction and automatic control type electric power reduction. Since the interior power supply is directly related to the living and business situation of the consumer, information is transmitted to the consumer for confirmation in principle each time. For the information transmission, various methods using portable terminals such as individually owned notebook computers or smartphones can be used, and there is no particular limitation on the methods. Subsequently, based on setting of the consumer, the electric power demand control is performed by two types of electric power reduction including the consumer participation type electric power reduction and the automatic control type electric power reduction. In general, since the electric power reduction of the interior power supply should be performed upon determining the situation each time, it is preferable that the customer participation type electric power reduction is selected as a criterion basic setting.

The electric power demand control of the air conditioner is performed as follows. It is highly possible that the predetermined interior space information and the position and movement information of the person 250 reflects the business state, which is the purpose of the person 250, and the sensitivity to the thermal sensation according to the movement of the person 250 and the communication between the persons 250. Therefore, it is highly possible that the grasp of the electric power demand at this time is to grasp the electric power demand that reflects the business state, which is the purpose of the person 250, and the sensitivity to the thermal sensation according to the movement of the person 250 and the communication between the persons 250. Such position information reflects the business state, which is the purpose of the person 250, and the sensitivity to the thermal sensation according to the movement of the person 250 and the communication between the persons 250, and the sensitivity to the thermal sensation is used to perform the electric power control.

Since the trend of the consumer is influenced in order to use the sensitivity to the thermal sensation, the control method is determined by collating implemented control contents with control result accumulation and the accumulated results, and the determination method is not particularly limited. The air conditioning control tries to be performed within a range that does not impair the comfort based on the living situation estimated based on the position information and the movement information of the person 250, such as known passive shimming air conditioning.

In the above example, although the accumulation data are searched for position information and movement information similar to the newly acquired position information and movement information of the person 250, it is also possible to search using only the position information.

According to the embodiment of the invention described above, since the electric power demand can be estimated based on the position information and the movement information of the person 250, it is possible to try the control within the range that does not impair the comfort. On the other hand, since it depends on the trend of the consumer in order to use the sensitivity to the thermal sensation, it is desirable to use a control validity verification method accompanying the control. The control validity verification method may be various existing methods. For example, a questionnaire of the person 250, a report on the thermal sensation using a personal terminal such as a business personal computer or a smartphone is effective.

The illumination is at the lowest priority as a control object. Since changes in the illuminance of the illumination and intermittent lighting impose a heavy burden on the resident, the control mainly involves the cooperation of the resident, such as encouraging the resident to move actively in the office space.

As described above, according to the present embodiment, by grasping the position and the movement of the resident in the interior space, the living and working situation of the person 250 can be grasped, and, by grasping the correlation with the electric power demand at that time, cooperation such as electric power demand reduction can be implemented without impairing the living and working situation of the person 250.

More specifically, according to the invention, it is possible to grasp the electric power demand of the consumer by grasping the living and working situation based on the position information of the consumer and electric power consumption at a stay time and at a stay position.

According to the invention, prediction of the electric power demand in the business department can be estimated based on the position information of the consumer and the electric power consumption at the stay time and at the stay position. Accordingly, it is possible to predict the electric power demand of the consumer and grasp the electric power demand for each time, and it is possible to cooperate with the electric power supply including the variable renewable energy (VRE).

According to the invention, it is possible to grasp the living and working situation of the consumer based on the position information of the consumer in the business department and the electric power consumption at the stay time and at the stay position. By the information transmission of the living and working situation of the consumer based on the invention, it is possible to provide a residential service added to the electric power supply and demand cooperation to the consumer.

According to the invention, it is possible to grasp the living and working situation of the consumer based on the position information of the consumer in the business department and the electric power consumption at the stay time and at the stay position. It is possible to provide information for diagnosing the validity of the living space along with the grasp of the living and working situation of the consumer, which can be useful for improving the living and working situation of the consumer.

The following is a summary of several representative examples of the invention.

(1) An energy management system (for example, the energy management system 200) includes a processor (for example, the processor 201) and a storage device (for example, the memory 202 and the storage device 203). The storage device stores, in association with each other, past position information (for example, the fourth group of data 504) indicating a past position of a person (for example, the person 250) in a target space (for example, the space 230) and electric power demand information (for example, third group of data 503) indicating an electric power demand in the target space when the past position information is acquired. The processor searches for the past position information similar to new position information indicating a newly acquired position of the person in the target space, and outputs control information for controlling the electric power demand in the target space based on the electric power demand information stored in the storage device corresponding to the past position information obtained by the search.

If the temperature in the space is too high or too low, which is difficult for a person in the space to accept, it is considered that the person himself/herself operates the air conditioner to correct the state. The same applies to the illumination device and other devices. That is, it is considered that the past electric power demand accumulated in the storage device can provide the thermal sensation and the brightness of the illumination accepted by the person in the space at that time.

A degree of the thermal sensation, the brightness of the illumination, and the usability of other devices that the person accepts are considered to depend on the action of the person. For example, as shown in FIG. 3, in the space in which there is a certain degree of correlation between the position and the action of the person, it is estimated that the persons at similar positions act similarly, so that the required thermal sensations are also estimated to be similar.

Therefore, as described above, by controlling the device in the target space based on the electric power demand stored corresponding to the position of the person similar to the newly acquired position of the person (for example, the position of the person in the current target space), it is possible to control the electric power demand within a range that the consumer can accept.

At this time, the processor may generate the control information for each category of the electric device. For example, the control information for controlling the electric power demand of the air conditioner may be generated based on the electric power demand information related to the air conditioner among the electric power demand information corresponding to the searched position information. The same applies to the illumination device and other electric devices. The processor may generate the control information for each area in the target space. For example, the control information for controlling the electric power demand in a certain area (for example, a conference room or a private room) may be generated based on the electric power demand information related to the same area among the electric power demand information corresponding to the searched position information.

(2) Here, the processor may output the control information for controlling a current electric power demand in the target space in a decreasing direction when the current electric power demand in the target space is larger than an electric power demand criterion specified based on the electric power demand information stored in the storage device corresponding to the past position information obtained by the search.

As described above, the electric power demand corresponding to position information obtained by the search is estimated to be acceptable to the person in the current target space. However, in general, since there are variations, when an electric power demand criterion may be specified and the current electric power demand is larger than the criterion, the current electric power demand may be controlled so as to approach the criterion. Accordingly, electric power saving within a range that can be accepted by the consumer can be achieved.

(3) Here, the electric power demand criterion may be an average value of the electric power demand calculated based on the electric power demand information stored in the storage device corresponding to the past position information obtained by the search.

However, the average value is an example, and, for example, a distribution of the electric power demand corresponding to the position information obtained by the search is statistically processed, and some statistical index can be used as the electric power demand criterion. This allows the electric power demand to be controlled to a value that is estimated to be particularly acceptable.

(4) Further, the processor, when receiving a request to reduce the electric power demand, may output the control information for controlling a current electric power demand in the target space in a decreasing direction within a range of the electric power demand specified based on the electric power demand information stored in the storage device corresponding to the past position information obtained by the search.

Specifically, for example, the processor may reduce the current electric power demand to the smallest electric power demand within the range of the variation in the electric power demand stored in the storage device corresponding to the past position information obtained by the search. Alternatively, the distribution of the electric power demand obtained by the search may be statistically processed and a reduction target may be determined based on a statistical index (for example, −1σ in a case where the distribution of the electric power demand is assumed to follow the normal distribution).

For example, when the energy management system is used to optimize the energy supply and demand in a region where the variable renewable energy is introduced, and when it is necessary to reduce the demand in accordance with the electric power supply, the above control can be performed to adjust the electric power demand within a range that can be accepted by the consumer to maintain a balance between supply and demand.

(5) Further, the past position information and the new position information may include information related to a movement of the person in the target space. Then, the processor may search the past position information for the position information related to a movement similar to a movement of the person specified based on the new position information of the person in the target space.

Accordingly, the past electric power demand corresponding to the situation closer to the current situation is searched, and it is possible to control the electric power demand more easily accepted by the consumer.

(6) Further, one or more electric devices may be installed in the target space. Then, the processor may output, as the control information, information for controlling the electric power consumption of the one or more electric devices.

(7) Here, the one or more electric devices may include at least one of one or more illumination devices (for example, the illumination device 232) that illuminate the target space, one or more air conditioners (for example, the air conditioner 233) that adjust a temperature of air in the target space, and one or more electric devices (for example, the PC 235 connected to the outlet 234) connected to one or more outlets of the target space. Further, the information for controlling the electric power consumption of the one or more electric devices may include at least one of information for controlling illumination intensity of the one or more illumination devices, information for controlling temperature adjustment intensity of the one or more air conditioners, and information for controlling electric power supply from the one or more outlets.

Accordingly, the electric power of various electric devices can be controlled and the desired electric power demand control can be implemented.

(8) The storage device may store, in association with each other, the past position information, the electric power demand information, and operation information (for example, the first group of data 501) indicating an operation situation of the target space when the past position information is acquired. Then, the processor may output the control information based on the electric power demand information corresponding to the operation information similar to the operation situation of the target space when the new position information is acquired among the electric power demand information stored in the storage device corresponding to the past position information obtained by the search.

Accordingly, the past electric power demand corresponding to the situation closer to the current situation is searched, and it is possible to control the electric power demand more easily accepted by the consumer.

(9) Here, the operation information may include at least one of a day of a week when the past position information is acquired, a weather condition when the past position information is acquired, and an event in the target space when the past position information is acquired.

For example, the processor may control the electric devices based on the electric power demand information acquired on the same day of the week, the same weather, and the same temperature when the new position information is acquired, or may control the electric device based on the electric power demand information acquired when an event of the same type as that performed when the new position information is acquired is performed.

Accordingly, the past electric power demand corresponding to the situation closer to the current situation is searched, and it is possible to control the electric power demand more easily accepted by the consumer.

(10) Further, the storage device may store, in association with each other, the past position information, the electric power demand information, and evaluation information indicating evaluation of the target space input by the person in the target space. Then, the processor may output the control information based on the electric power demand information corresponding to the evaluation information satisfying a predetermined condition among the electric power demand information stored in the storage device corresponding to the past position information obtained by the search.

For example, the person in the target space may input the evaluation of the current thermal sensation (or a specific period in the past), the brightness of the illumination, the usability of other electric devices, and the like into the energy management system. The storage device may store such evaluation information in association with the position information and electric power demand information acquired at that time, and may search for only the position information and the electric power demand information corresponding to high evaluation exceeding a predetermined criterion.

Accordingly, it is possible to control the electric power demand that is more likely to be accepted by the consumer.

(11) The storage device may store, in association with each other, the past position information, the electric power demand information, and biological information (for example, biological information acquired by the personal information sensor 238) of the person in the target space. Then, the processor may output the control information based on the electric power demand information corresponding to the biological information satisfying a predetermined condition in the electric power demand information stored in the storage device corresponding to the past position information obtained by the search.

For example, the current electric power demand may be controlled to be similar to the electric power demand when it is estimated that the state of the person is good based on the biological information. For example, when the biological information includes information on a pulse of a person, a stress state of the person may be estimated based on the pulse, and the control may be performed based on the electric power demand in a state where the stress is low.

Accordingly, it is possible to control the electric power demand that is more likely to be accepted by the consumer.

(12) Further, the processor may output information indicating the electric power demand in the target space to be a control target corresponding to the newly acquired position based on the electric power demand information stored in the storage device corresponding to the past position information obtained by the search.

Accordingly, it is possible to feed back the information of the control to be appropriately estimated to the consumer, and use the information for control by the customer himself/herself thereafter.

(13) Further, the processor may output information urging a review of a layout of the target space when a person position disorder included in the past position information stored in the storage device is larger than a predetermined criterion.

In order to perform the energy management of the invention, it is desirable that there is a certain degree of correlation between the position of the person in the target space and the action of the person (for example, the PC work is often performed when the person is in a private room). However, for example, when there is no bias in the distribution of the person position in the target space acquired in a sufficiently long past period, and the persons are evenly distributed in the entire target space (that is, the person position disorder is large), it is considered that the correlation between the position and the action of the person is not strong enough. In this case, information may be output that urges a change in the layout in the space (for example, the arrangement of furniture) so that the correlation between the position and the action of the person increases.

Accordingly, it is possible to generate a space in which the electric power demand can be controlled more appropriately.

(14) The energy management system may include a communication device (for example, the communication device 206) connected to the processor, a network (for example, the network 220) connected to the communication device, a position sensor (for example, the position sensor 231) connected to the network and configured to measure the position of the person in the target space, and at least one of an illumination device (for example, the illumination device 232) capable of controlling illumination intensity based on the control information, an air conditioner (for example, the air conditioner 233) capable of controlling temperature adjustment intensity based on the control information, and an outlet (for example, the outlet 234) capable of controlling electric power supply based on the control information, which are connected to the network.

Accordingly, a system for controlling the electric power demand based on the sensor information is implemented.

The invention is not limited to the embodiments described above, and includes various modifications. For example, the embodiments described above are described in detail for better understanding of the invention. The invention is not necessarily limited to embodiments including all configurations described above. A part of configurations of one embodiment can be replaced with configurations of another embodiment. Alternatively, configurations of an embodiment can be added to configurations of another embodiment. In addition, a part of the configuration of each embodiment may be added, deleted, or replaced with another configuration.

Configurations, functions, processing units, processing methods and the like described above may be partially or entirely implemented by hardware such as through design using an integrated circuit. The configurations, functions, and the like may also be implemented by software by means of interpreting and executing a program, by a processor, for implementing their respective functions. Information such as a program, a table, or a file for implementing each function can be stored in a storage device such as a nonvolatile semiconductor memory, a Hard Disk Drive, and a Solid State Drive (SSD), or a computer readable non-transitory data storage medium such as an IC card, an SD card, and a DVD.

Control lines and information lines show those considered to be necessary for description, and not all of the control lines and the information lines are necessarily shown on the product. In practice, it may be considered that almost all of the configurations are connected to each other.

What is claimed is:

1. An energy management system, comprising:
a processor; and
a storage device, wherein
the storage device stores, in association with each other, past position information indicating a past position of a person in a target space and electric power demand information indicating an electric power demand in the target space when the past position information is acquired, and
the processor
searches for the past position information similar to new position information indicating a newly acquired position of the person in the target space, and
outputs control information for controlling the electric power demand in the target space based on the electric power demand information stored in the storage device corresponding to the past position information obtained by the search.

2. The energy management system according to claim 1, wherein
the processor outputs the control information for controlling a current electric power demand in the target space in a decreasing direction when the current electric power demand in the target space is larger than an electric power demand criterion specified based on the electric power demand information stored in the storage device corresponding to the past position information obtained by the search.

3. The energy management system according to claim 2, wherein
the electric power demand criterion is an average value of the electric power demand calculated based on the electric power demand information stored in the storage device corresponding to the past position information obtained by the search.

4. The energy management system according to claim 1, wherein
the processor, when receiving a request to reduce the electric power demand, outputs the control information for controlling a current electric power demand in the target space in a decreasing direction within an electric power demand range specified based on the electric power demand information stored in the storage device corresponding to the past position information obtained by the search.

5. The energy management system according to claim 1, wherein
the past position information and the new position information include information related to a movement of the person in the target space, and
the processor searches the past position information for position information related to a movement similar to a movement of the person specified based on the new position information of the person in the target space.

6. The energy management system according to claim 1, wherein
one or more electric devices are installed in the target space, and
the processor outputs, as the control information, information for controlling electric power consumption of the one or more electric devices.

7. The energy management system according to claim 6, wherein
the one or more electric devices include at least one of one or more illumination device that illuminate the target space, one or more air conditioners that adjust a temperature of air in the target space, and one or more electric devices connected to one or more outlets of the target space, and
the information for controlling the electric power consumption of the one or more electric devices includes at least one of information for controlling illumination intensity of the one or more illumination devices, information for controlling temperature adjustment intensity of the one or more air conditioners, and information for controlling electric power supply electric power from the one or more outlets.

8. The energy management system according to claim 1, wherein
the storage device stores, in association with each other, the past position information, the electric power demand information, and operation information indicating an operation situation of the target space when the past position information is acquired, and
the processor outputs the control information based on the electric power demand information corresponding to the operation information similar to the operation situation of the target space when the new position information is acquired among the electric power demand information stored in the storage device corresponding to the past position information obtained by the search.

9. The energy management system according to claim 8, wherein
the operation information includes at least one of a day of a week when the past position information is acquired, a weather condition when the past position information is acquired, and an event in the target space when the past position information is acquired.

10. The energy management system according to claim 1, wherein
the storage device stores, in association with each other, the past position information, the electric power demand information, and evaluation information indicating evaluation of the target space input by the person in the target space, and
the processor outputs the control information based on the electric power demand information corresponding to the evaluation information satisfying a predetermined condition among the electric power demand information stored in the storage device corresponding to the past position information obtained by the search.

11. The energy management system according to claim 1, wherein
the storage device stores, in association with each other, the past position information, the electric power demand information, and biological information of the person in the target space, and
the processor outputs the control information based on the electric power demand information corresponding to the biological information satisfying a predetermined condition among the electric power demand information stored in the storage device corresponding to the past position information obtained by the search.

12. The energy management system according to claim 1, wherein
the processor outputs information indicating the electric power demand in the target space to be a control target corresponding to the newly acquired position based on the electric power demand information stored in the storage device corresponding to the past position information obtained by the search.

13. The energy management system according to claim 1, wherein
the processor outputs information urging a review of a layout of the target space when a person position disorder included in the past position information stored in the storage device is larger than a predetermined criterion.

14. The energy management system according to claim 1, further comprising:
a communication device connected to the processor;
a network connected to the communication device;
a position sensor connected to the network and configured to measure the position of the person in the target space; and
at least one of an illumination device capable of controlling illumination intensity based on the control information, an air conditioner capable of controlling temperature adjustment intensity based on the control information, and an outlet capable of controlling electric power supply based on the control information, which is connected to the network.

15. An energy management method executed by an energy management system including a processor and a storage device, and the storage device storing, in association with each other, past position information indicating a past position of a person in a target space and electric power demand information indicating an electric power demand in the target space when the past position information is acquired, the energy management method comprising:
a procedure in which the processor searches for the past position information similar to new position information indicating a newly acquired position of the person in the target space, and
a procedure in which the processor outputs control information for controlling the electric power demand in the target space based on the electric power demand information stored in the storage device corresponding to the past position information obtained by the search.
